SEQUENCE LISTING (151 sequences)

SEQ ID NO: 1 - DNA, 3399 bp, Homo sapiens
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVIVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - protein, 9 aa, Homo sapiens
RPAEEATSL

SEQ ID NO: 4 - protein, 9 aa, Homo sapiens
RPSFLLSSL

SEQ ID NO: 5 - protein, 9 aa, Homo sapiens
DPRRLVQLL

SEQ ID NO: 6 - protein, 9 aa, Homo sapiens
FVRACLRRL

SEQ ID NO: 7 - protein, 9 aa, Homo sapiens
AGRNMRRKL

SEQ ID NO: 8 - protein, 9 aa, Homo sapiens
LPGTTLTAL

SEQ ID NO: 9 - protein, 9 aa, Homo sapiens
LPSDFKTIL

SEQ ID NO: 10 - protein, 9 aa, Homo sapiens
ILAKFLHWL

SEQ ID NO: 11 - protein, 9 aa, Homo sapiens
RLVDDFLLV

SEQ ID NO: 12 - protein, 9 aa, Homo sapiens
YLFFYRKSV

SEQ ID NO: 13 - protein, 13 aa, Homo sapiens
TPPAYRPPNAPIL

SEQ ID NO: 14 - protein, 9 aa, Homo sapiens
RLREEILAK

SEQ ID NO: 15 - protein, 9 aa, Homo sapiens
KLTRHRVTY

SEQ ID NO: 16 - protein, 9 aa, Homo sapiens
AVRIRGKSY

SEQ ID NO: 17 - protein, 9 aa, Homo sapiens
CVRRYAVVQ

SEQ ID NO: 18 - protein, 9 aa, Homo sapiens
RVGDDVLVH

SEQ ID NO: 19 - protein, 9 aa, Homo sapiens
KLFGVLRLK

SEQ ID NO: 20 - protein, 9 aa, Homo sapiens
ALRGSGAWG

SEQ ID NO: 21 - protein, 9 aa, Homo sapiens
ELVARVLQR

SEQ ID NO: 22 - protein, 9 aa, Homo sapiens
RLPRLPQRY

SEQ ID NO: 23 - protein, 9 aa, Homo sapiens
AVTPAAGVC

SEQ ID NO: 24 - protein, 9 aa, Homo sapiens
RLREEILAK

SEQ ID NO: 25 - protein, 9 aa, Homo sapiens
YVTETTFQK

SEQ ID NO: 26 - protein, 9 aa, Homo sapiens
KLFGVLRLK

SEQ ID NO: 27 - protein, 9 aa, Homo sapiens
KTFLRTLVR

SEQ ID NO: 28 - protein, 9 aa, Homo sapiens
SVYVVELLR

SEQ ID NO: 29 - protein, 9 aa, Homo sapiens
RVLQRLCER

SEQ ID NO: 30 - protein, 9 aa, Homo sapiens
QTVCTNIYK

SEQ ID NO: 31 - protein, 9 aa, Homo sapiens
RVLQRLCER

SEQ ID NO: 32 - protein, 9 aa, Homo sapiens
KTFLRTLVR

SEQ ID NO: 33 - protein, 9 aa, Homo sapiens
KILLLQAYR

SEQ ID NO: 34 - protein, 9 aa, Homo sapiens
SVYVVELLR

SEQ ID NO: 35 - protein, 9 aa, Homo sapiens
SVRDCAWLR

SEQ ID NO: 36 - protein, 9 aa, Homo sapiens
DVLVHLLAR

SEQ ID NO: 37 - protein, 9 aa, Homo sapiens
EVRQHREAR

SEQ ID NO: 38 - protein, 9 aa, Homo sapiens
SVLNYERAR

SEQ ID NO: 39 - protein, 9 aa, Homo sapiens
VVGARTFRR

SEQ ID NO: 40 - protein, 9 aa, Homo sapiens
YVVGARTFR

SEQ ID NO: 41 - protein, 9 aa, Homo sapiens
RVLQRLCER

SEQ ID NO: 42 - protein, 9 aa, Homo sapiens
SVYVVELLR

SEQ ID NO: 43 - protein, 9 aa, Homo sapiens
LVCVPWDAR

SEQ ID NO: 44 - protein, 9 aa, Homo sapiens
SVRDCAWLR

SEQ ID NO: 45 - protein, 9 aa, Homo sapiens
YVPLLGSLR

SEQ ID NO: 46 - protein, 9 aa, Homo sapiens
ELVARVLQR

SEQ ID NO: 47 - protein, 9 aa, Homo sapiens
EVIASIIKP

SEQ ID NO: 48 - protein, 9 aa, Homo sapiens
GAWGLLLRR

SEQ ID NO: 49 - protein, 9 aa, Homo sapiens
LSLQELTWK

SEQ ID NO: 50 - protein, 9 aa, Homo sapiens
QTVCTNIYK

SEQ ID NO: 51 - protein, 9 aa, Homo sapiens
DEALGGTAF

SEQ ID NO: 52 - protein, 9 aa, Homo sapiens
VELLRSFFY

SEQ ID NO: 53 - protein, 9 aa, Homo sapiens
REVLPLATF

SEQ ID NO: 54 - protein, 9 aa, Homo sapiens
WDTPCPPVY

SEQ ID NO: 55 - protein, 9 aa, Homo sapiens
EEDTDPRRL

SEQ ID NO: 56 - protein, 9 aa, Homo sapiens
KEQLRPSFL

SEQ ID NO: 57 - protein, 9 aa, Homo sapiens
REVLPLATF

SEQ ID NO: 58 - protein, 9 aa, Homo sapiens
CERGAKNVL

SEQ ID NO: 59 - protein, 9 aa, Homo sapiens
REAGVPLGL

SEQ ID NO: 60 - protein, 9 aa, Homo sapiens
AEHRLREEI

SEQ ID NO: 61 - protein, 9 aa, Homo sapiens
REEILAKFL

SEQ ID NO: 62 - protein, 9 aa, Homo sapiens
VELLRSFFY

SEQ ID NO: 63 - protein, 9 aa, Homo sapiens
PEYGCVVNL

SEQ ID NO: 64 - protein, 9 aa, Homo sapiens
DEALGGTAF

SEQ ID NO: 65 - protein, 9 aa, Homo sapiens
YERARRPGL

SEQ ID NO: 66 - protein, 9 aa, Homo sapiens
LEAAANPAL

SEQ ID NO: 67 - protein, 9 aa, Homo sapiens
REAGVPLGL

SEQ ID NO: 68 - protein, 9 aa, Homo sapiens
LEAAANPAL

SEQ ID NO: 69 - protein, 9 aa, Homo sapiens
REEILAKFL

SEQ ID NO: 70 - protein, 9 aa, Homo sapiens
EEDTDPRRL

SEQ ID NO: 71 - protein, 9 aa, Homo sapiens
YERARRPGL

SEQ ID NO: 72 - protein, 9 aa, Homo sapiens
KEQLRPSFL

SEQ ID NO: 73 - protein, 9 aa, Homo sapiens
CERGAKNVL

SEQ ID NO: 74 - protein, 9 aa, Homo sapiens
QELTWKMSV

SEQ ID NO: 75 - protein, 9 aa, Homo sapiens
AEVRQHREA

SEQ ID NO: 76 - protein, 9 aa, Homo sapiens
EEATSLEGA

SEQ ID NO: 77 - protein, 9 aa, Homo sapiens
QETSPLRDA

SEQ ID NO: 78 - protein, 9 aa, Homo sapiens
CERAWNHSV

SEQ ID NO: 79 - protein, 9 aa, Homo sapiens
REKPQGSVA

SEQ ID NO: 80 - protein, 9 aa, Homo sapiens
REAGVPLGL

SEQ ID NO: 81 - protein, 9 aa, Homo sapiens
LEAAANPAL

SEQ ID NO: 82 - protein, 9 aa, Homo sapiens
YAETKHFLY

SEQ ID NO: 83 - protein, 9 aa, Homo sapiens
ISDTASLCY

SEQ ID NO: 84 - protein, 9 aa, Homo sapiens
DTDPRRLVQ

SEQ ID NO: 85 - protein, 11 aa, Homo sapiens
AQDPPPELYDF

SEQ ID NO: 86 - protein, 9 aa, Homo sapiens
LTDLQPYMR

SEQ ID NO: 87 - protein, 9 aa, Homo sapiens
QSDYSSYAR

SEQ ID NO: 88 - protein, 9 aa, Homo sapiens
STLLCSLCY

SEQ ID NO: 89 - protein, 9 aa, Homo sapiens
STLTDLQPY

SEQ ID NO: 90 - protein, 9 aa, Homo sapiens
RTLEVQSDY

SEQ ID NO: 91 - protein, 9 aa, Homo sapiens
VTETTFQKN

SEQ ID NO: 92 - protein, 9 aa, Homo sapiens
KALFSVLNY

SEQ ID NO: 93 - protein, 9 aa, Homo sapiens
KLTRHRVTY

SEQ ID NO: 94 - protein, 9 aa, Homo sapiens
EVQSDYSSY

SEQ ID NO: 95 - protein, 9 aa, Homo sapiens
EVQSDYSSY

SEQ ID NO: 96 - protein, 9 aa, Homo sapiens
YVVELLRSF

SEQ ID NO: 97 - protein, 9 aa, Homo sapiens
EVIASIIKP

SEQ ID NO: 98 - protein, 9 aa, Homo sapiens
ETTFQKNRL

SEQ ID NO: 99 - protein, 9 aa, Homo sapiens
VVIEQSSSL

SEQ ID NO: 100 - protein, 9 aa, Homo sapiens
ETIFLGSRP

SEQ ID NO: 101 - protein, 9 aa, Homo sapiens
DVLVHLLAR

SEQ ID NO: 102 - protein, 9 aa, Homo sapiens
RVISDTASL

SEQ ID NO: 103 - protein, 9 aa, Homo sapiens
EATSLEGAL

SEQ ID NO: 104 - protein, 9 aa, Homo sapiens
ETKHFLYSS

SEQ ID NO: 105 - protein, 9 aa, Homo sapiens
TYVPLLGSL

SEQ ID NO: 106 - protein, 9 aa, Homo sapiens
CYGDMENKL

SEQ ID NO: 107 - protein, 9 aa, Homo sapiens
AYQVCGPPL

SEQ ID NO: 108 - protein, 9 aa, Homo sapiens
VYGFVRACL

SEQ ID NO: 109 - protein, 9 aa, Homo sapiens
VYAETKHFL

SEQ ID NO: 110 - protein, 9 aa, Homo sapiens
AYRFHACVL

SEQ ID NO: 111 - protein, 9 aa, Homo sapiens
RYWQMRPLF

SEQ ID NO: 112 - protein, 9 aa, Homo sapiens
DYVVGARTF

SEQ ID NO: 113 - protein, 9 aa, Homo sapiens
RFIPKPDGL

SEQ ID NO: 114 - protein, 9 aa, Homo sapiens
DFLLVTPHL

SEQ ID NO: 115 - protein, 9 aa, Homo sapiens
RFHACVLQL

SEQ ID NO: 116 - protein, 9 aa, Homo sapiens
RFLRNTKKF

SEQ ID NO: 117 - protein, 9 aa, Homo sapiens
RRFLRNTKK

SEQ ID NO: 118 - protein, 9 aa, Homo sapiens
RRLVETIFL

SEQ ID NO: 119 - protein, 9 aa, Homo sapiens
RRDGLLLRL

SEQ ID NO: 120 - protein, 9 aa, Homo sapiens
RREKRAERL

SEQ ID NO: 121 - protein, 9 aa, Homo sapiens
KRAERLTSR

SEQ ID NO: 122 - protein, 9 aa, Homo sapiens
RRRGGSASR

SEQ ID NO: 123 - protein, 9 aa, Homo sapiens
RRLPRLPQR

SEQ ID NO: 124 - protein, 9 aa, Homo sapiens
RRYAVVQKA

SEQ ID NO: 125 - protein, 9 aa, Homo sapiens
LRFMCHHAV

SEQ ID NO: 126 - protein, 9 aa, Homo sapiens
RRLGPQGWR

SEQ ID NO: 127 - protein, 9 aa, Homo sapiens
RRKLFGVLR

SEQ ID NO: 128 - protein, 9 aa, Homo sapiens
QRYWQMRPL

SEQ ID NO: 129 - protein, 9 aa, Homo sapiens
SRSLPLPKR

SEQ ID NO: 130 - protein, 9 aa, Homo sapiens
GRTRGPSDR

SEQ ID NO: 131 - protein, 9 aa, Homo sapiens
RRLVPPGLW

SEQ ID NO: 132 - protein, 9 aa, Homo sapiens
RRYAVVQKA

SEQ ID NO: 133 - protein, 9 aa, Homo sapiens
LRLKCHSLF

SEQ ID NO: 134 - protein, 9 aa, Homo sapiens
ARGGPPEAF

SEQ ID NO: 135 - protein, 9 aa, Homo sapiens
LREEILAKF

SEQ ID NO: 136 - protein, 9 aa, Homo sapiens
VRSLLRSHY

SEQ ID NO: 137 - protein, 9 aa, Homo sapiens
ARRLVETIF

SEQ ID NO: 138 - protein, 9 aa, Homo sapiens
LRPIVNMDY

SEQ ID NO: 139 - protein, 9 aa, Homo sapiens
RREKRAERL

SEQ ID NO: 140 - protein, 9 aa, Homo sapiens
RRDGLLLRL

SEQ ID NO: 141 - protein, 9 aa, Homo sapiens
RRLVETIFL

SEQ ID NO: 142 - protein, 9 aa, Homo sapiens
QHREARPAL

SEQ ID NO: 143 - protein, 9 aa, Homo sapiens
RHRVTYVPL

SEQ ID NO: 144 - protein, 9 aa, Homo sapiens
VHLLARCAL

SEQ ID NO: 145 - protein, 9 aa, Homo sapiens
SHYREVLPL

SEQ ID NO: 146 - protein, 9 aa, Homo sapiens
EHRLREEIL

SEQ ID NO: 147 - protein, 9 aa, Homo sapiens
AHLQETSPL

SEQ ID NO: 148 - protein, 9 aa, Homo sapiens
SHVSTLTDL

SEQ ID NO: 149 - protein, 9 aa, Homo sapiens
CHQAFLLKL

SEQ ID NO: 150 - protein, 9 aa, Homo sapiens
HASGPRRRL

SEQ ID NO: 151 - protein, 9 aa, Homo sapiens
AHGHVRKAF

(19) United States
(12) Patent Application Publication
(10) Pub. No.: US 2009/0202499 A1
(43) Pub. Date: Aug. 13, 2009

(54) Human Telomerase Reverse Transcriptase Peptides

(75) Inventors: Maurizio Zanetti, La Jolla, CA (US); Xochitl Cortez-Gonzalez, La Jolla, CA (US)

Correspondence Address: MORRISON & FOERSTER LLP, 425 MARKET STREET, SAN FRANCISCO, CA, 94105-2482, US

(73) Assignee: THE REGENTS OF THE UNIVERSITY OF CALIFORNIA, Oakland, CA (US)

(21) Appl. No.: 12/161,552

(22) PCT Filed: Jan. 19, 2007
(86) PCT No.: PCT/US2007/001587

§ 371 Date: Jan. 7, 2009

Related U.S. Application Data
(60) Provisional application No. 60/761,009, filed on Jan. 19, 2006.

(51) Int. Cl. A61K 35/12; A61K 38/43; C12Q 1/48; C12N 9/10; C12N 5/02

(52) U.S. Cl. 424/93.7; 424/94.5; 435/15; 435/193; 435/375

(57) ABSTRACT

Tumor antigens can be categorized as tumor type specific or common. Telomerase reverse transcriptase (TRT) is the first bona fide common tumor antigen. While several 9mer peptides of the human TRT (hTRT) have been identified for HLA-A2, the most prevalent (˜50%) HLA type in humans, little information exists on peptides for the remaining HLA types. As described herein, a multi-step approach was taken to select and characterize a panel of HLA-B79mer peptides as candidate immunogens. Specifically, several of algorithm based predictions, in vivo immunization of HLA-B7 transgenic mice, in vitro immunization of human blood lymphocytes, in vivo processing and supertype binding were employed to identify HLA-B7-restricted epitopes in hTRT. A correlation between in vivo immunogenicity and actual HLA-B7 binding avidity was found for the seven predicted peptides. Furthermore, endogenous processing was found to correlate with in vitro immunogenicity in human PBMC and HLA-B7 supertype binding.

The invention was made in part with government support from the National Institutes of Health Grant Nos. RO1CA084062 and 5T32GM008666-07, and from the National Science Foundation Grant No. 9978892. As such, the Unites States government may have certain rights in the invention.

### FIELD OF THE INVENTION

The present invention is directed to cancer immunotherapy and studies thereof. In particular, the present invention provides compositions and methods for inducing cytotoxic T lymphocyte responses to cells that present human telomerase reverse transcriptase peptides. In addition, the present invention provides tools for identifying immunogenic human telomerase reverse transcriptase peptides.

### BACKGROUND OF THE INVENTION

Telomerase is a ribonucleoprotein that mediates RNA-dependent synthesis of telomeric DNA (1). Maintenance of a constant telomere length ensures chromosomal stability, prevents cells from aging, and confers immortality (2-4). In vitro studies show that the long-term ectopic expression of human telomerase reverse transcriptase (hTRT) in normal fibroblasts is sufficient for immortalization (5), and the expression of hTRT in combination with two oncogenes (SV40 T antigen and Ras) promotes tumor transformation in normal human epithelial and fibroblast cell lines (6). Thus, although telomerase per se is not tumorigenic, it plays a direct role in oncogenesis by allowing pre-cancerous cells to proliferate continuously and become immortal.

Studies of human cancer cells have shown a striking high expression (>85%) of telomerase activity in tumors of different histological origin and type (7, 8). In contrast, normal tissues display little or no telomerase activity (8, 9). For these reasons hTRT is considered the prototype common tumor antigen (10). To date numerous in vitro studies have been published demonstrating that hTRT peptides can be used to expand CD8 T cell precursors and generate cytotoxic T lymphocytes (CTL) in human peripheral blood mononuclear cells (PBMC) (11-15). Furthermore, several Phase 1 trials have also been completed proving that specific CD8 T cell responses can be induced in vivo (16-19) in cancer patients.

T lymphocytes recognize antigens through the intermediary of molecules of the major histocompatibility complex (MHC) or human leukocyte antigen (HLA), a polymorphic system composed of several hundred molecules (“MHC restriction”). CD8 T cells recognize antigen presented through MHC Class I molecules expressed at the surface of every cell after antigen peptides have been processed inside the cell and exported to the cell surface through the endogenous pathway (20). Under normal circumstances, MHC Class I molecules present a broad variety of peptides, mainly the product of processing of endogenous proteins. Upon infection by microbial pathogens or tumor transformation, peptides are generated that once complexed with the MHC molecules of an antigen presenting cell (APC) can activate CD8 T cells and induce CTL responses. However, since the MHC system is highly polymorphic among the human population, it requires that the immunogenicity of antigen peptides be studied in relation to each HLA molecule. An alternative and simpler approach is to test antigen peptides in relation to HLA alleles grouped into large supertype families (21). A HLA supertype is defined by the ability of a peptide to bind multiple HLA molecules (supermotif). The HLA alleleic variants that bind peptides possessing a particular HLA supertmotif are referred to as HLA supertype. The HLA-B7 supertype includes the B*0702, B*3501-03, B*51, B*5301, B*5401, B*0703-05, B*1508, B*5501-02, B*5601-02, B*6701 and B*7801 alleles. These HLA molecules share a peptide binding specificity for P in position 2 and a hydrophobic aliphatic (A, L, I, M, or V) or aromatic (F, W, or Y) residue at the C-terminal position (22).

To date specific information on the immunogenicity hTRT peptides is limited to one MHC allele (HLA-A*0201) with only initial reports on the HLA-A3 (13) and HLA-A24 (23) types, respectively. Although HLA-A*0201 is the most frequent in the human population (95% of HLA-A2 type which is itself expressed in 50% of the Caucasian population (24-26)) immunogenic peptides for an equally large segment of the human population need to be identified. The goal of the work presented here was to identify immunogenic hTRT peptides restricted by HLA-B*0702 molecule, which is the most prevalent allele within the HLA-B7 type accounting for ˜8.6% of the Caucasian population (27).

### SUMMARY OF THE INVENTION

The present invention is directed to cancer immunotherapy and studies thereof. In particular, the present invention provides compositions and methods for inducing cytotoxic T lymphocyte responses to cells that present human telomerase reverse transcriptase (hTRT) peptides. In addition, the present invention provides compositions and methods for identifying immunogenic hTRT peptides presented by the most frequently expressed major histocompatibility complex (MHC) class I types and supertypes. Specifically, in some embodiments the present invention provides compositions and methods comprising at least one human leukocyte antigen (HLA)-B7-restricted hTRT peptide. In further embodiments, compositions and methods comprising one or more of an HLA-A3-restricted hTRT peptide, an HLA-A2-restricted hTRT peptide, an HLA-A24-restricted hTRT peptide, an HLA-B44-restricted hTRT peptide, an HLA-A1-restricted hTRT peptide, and an HLA-B27-restricted hTRT peptide, are provided.

In still further embodiments, the present invention provides methods and compositions comprising an immunoglobulin molecule comprising an HLA class I restricted hTRT epitope inserted therein (e.g., recombinant antibody comprising an hTRT epitope expressed as part of a heavy or light chain variable region). The teaching of the production of antigenized antibodies can be found for instance in U.S. Pat. Nos. 5,658,762, 5,583,202, and 5,508,386 to Zanetti et al. (herein incorporated by reference in their entirety).

In addition, in some embodiments the present invention provides methods and compositions for inducing a cytotoxic T lymphocyte response, comprising a first HLA Class I restricted hTRT peptide, wherein the first peptide is an HLA-A2-restricted hTRT peptide, and a second HLA Class I restricted hTRT peptide, wherein the second peptide comprises one or more of an HLA-B7-restricted hTRT peptide, an HLA-A3-restricted hTRT peptide, an HLA-A24-restricted hTRT peptide, an HLA-B44-restricted hTRT peptide, an HLA-A1-restricted hTRT peptide, and an HLA-B27-restricted hTRT. The teaching of HLA-A*0201-restricted hTRT peptides can be found for instance in U.S. Publication No. 20040086518 of Zanetti, and PCT Publication No. WO 00/25813 of Nadler et al. (both herein incorporated by reference in their entirety). In some embodiments, the HLA-A2-restricted hTRT peptide is selected from the group consisting of p540 (ILAKFLHWL, set forth as SEQ ID NO:10) and p865 (RLVDDFLLV, set forth as SEQ ID NO:11). In still further embodiments, the HLA-A2-restricted hTRT peptide comprises a modification which increases its binding affinity for HLA-A2 (e.g., p572Y, YLFFYRKSV, set forth as SEQ ID NO: 12). Further teaching of HLA-A2-restricted peptides, with and without modifications for increasing their binding affinity for HLA-A2 can be found in Minev et al., Proc Natl Acad Sci USA, 97:4796-4801, 2000; and Hernandez et al., Proc Natl Acad Sci USA, 99:12275-12280, 2002 (both herein incorporated by reference in their entirety).

Specifically, the present invention provides compositions for induction of a cytotoxic T lymphocyte response, comprising: at least one HLA-B7-restricted human telomerase reverse transcriptase (TRT) peptide from nine to twelve amino acid residues in length (e.g., 9, 10, 11 or 12 residues). In some embodiments, the HLA-B7 is selected from the group consisting of HLA-B*0702, HLA-B*3501, HLA-B*3502, HLA-B*3503, HLA-B*5101, HLA-B*5301, HLA-B*5401, HLA-B*0703, HLA-B*0704, HLA-B*0705, HLA-B*1508, HLA-B*5501, HLA-B*5502, HLA-B*5601, HLA-B*5602, HLA-B*6701, HLA-B*7801, and HLA-B*0801. In some preferred embodiments, the at least one TRT peptide consists of a sequence selected from the group consisting of SEQ ID NO:3 (p277), SEQ ID NO:4 (p342), SEQ ID NO:6 (p464), SEQ ID NO:8 (p1107), and SEQ ID NO:9 (p1123). In further embodiments, the composition also comprises a helper peptide, wherein the TRT peptide is not conjugated to the helper peptide. In an exemplary embodiments, the helper peptide corresponds to residues 128 to 140 of the hepatitis B core antigen (TPPAYRPPNAPIL, set forth as SEQ ID NO:13). In still further embodiments, the composition also comprises an adjuvant. In some embodiments, the compositions further comprise a physiologically acceptable carrier, which in preferred embodiments is a mammalian cell (e.g., antigen presenting cells such as a dendritic cell, a B lymphocyte or a macrophage having a TRT peptide bound to HLA class I molecules on the cell surface). Also provided are compositions in which the TRT peptide comprises a modification to enhance binding to HLA-B7. In some embodiments, the modification is a substitution of the first residue of a TRT nonamer with a tyrosine). In some preferred embodiments, the TRT peptide is a synthetic peptide.

Moreover, the present invention provides methods for inducing or enhancing a CTL response against target cells expressing human TRT and HLA-B7, comprising: harvesting leucocytes expressing HLA-B7; pulsing the leukocytes with a composition comprising an HLA-B7 restricted human TRT peptide from nine to twelve amino acid residues in length (e.g., 9, 10, 11 or 12 residues); and contacting target cells expressing human TRT and HLA-B7 with the pulsed leucocytes. In some embodiments, the contacting is accomplished in vitro or ex vivo while in alternative embodiments the contacting is accomplished in vivo. In some embodiments, the HLA-B7 is selected from the group consisting of HLA-B*0702, HLA-B*3501, HLA-B*3502, HLA-B*3503, HLA-B*5101, HLA-B*5301, HLA-B*5401, HLA-B*0703, HLA-B*0704, HLA-B*0705, HLA-B*1508, HLA-B*5501, HLA-B*5502, HLA-B*5601, HLA-B*5602, HLA-B*6701, HLA-B*7801, and HLA-B*0801. In some preferred embodiments, the at least one TRT peptide consists of a sequence selected from the group consisting of SEQ ID NO:3 (p277), SEQ ID NO:4 (p342), SEQ ID NO:6 (p464), SEQ ID NO:8 (p 1107), and SEQ ID NO:9 (p 1123).

Additionally, the present invention provides methods for screening HLA class I-restricted human telomerase reverse transcriptase (TRT) peptides, comprising: a) using an algorithm to identify a human telomerase reverse transcriptase (TRT) peptide sequence in the full length TRT protein sequence that corresponds to a canonical HLA class I motif and comprises at least nine amino acid residues; b) testing HLA class I binding of the TRT peptide sequence by measuring HLA class I binding or stabilization in comparison to a reference peptide; and c) assessing immunogenicity of the TRT peptide sequence by measuring induction of TRT peptide-reactive cytotoxic T lymphocytes (CTL) of an HLA class I-positive subject. In some embodiments, the HLA class I-positive subject was immunized with a candidate human TRT vaccine (e.g., immunogenic composition) prior to the assessing of step c). In some preferred embodiments, the human TRT vaccine comprises human TRT DNA. In other preferred embodiments, the human TRT vaccine comprises a recombinant microorganism engineered to express human TRT. In further embodiments, the human TRT vaccine comprises a TRT peptide from nine to twelve amino acid residues in length (e.g., 9, 10, 11 or 12 residues), which in some embodiments is formulated with a liposome. In preferred methods, HLA class I is HLA-B7, while in particularly preferred embodiments the HLA-B7 binding comprises HLA-B*0702 binding, and one or more of HLA-B*3501, HLA-B*3502, HLA-B*3503, HLA-B*5101, HLA-B*5301, HLA-B*5401, HLA-B*0703, HLA-B*0704, HLA-B*0705, HLA-B*1508, HLA-B*5501, HLA-B*5502, HLA-B*5601, HLA-B*5602, HLA-B*6701, HLA-B*7801, and HLA-B*0801 binding. In alternative embodiments, the HLA class I is selected from the group consisting of HLA-A3, HLA-A24, HLA-B44, HLA-A1 and HLA-B27. In some embodiments, the HLA class I-positive subject is a transgenic mouse.

Also provided by the present invention are compositions for induction of a cytotoxic T lymphocyte response, comprising: at least one HLA class I-restricted human telomerase reverse transcriptase (hTRT) peptide from nine to twelve amino acid residues in length, wherein the hTRT peptide comprises one or more of an HLA-A3-restricted hTRT peptide, an HLA-A24-restricted hTRT peptide, an HLA-B44-restricted hTRT peptide, an HLA-A1-restricted hTRT peptide, and an HLA-B27-restricted hTRT.

### DESCRIPTION OF THE FIGURES

FIG. 1. In vivo CTL responses against p277, p342, p444, p464, p966, p1107 and p1123 in HLA-B7 Tg mice. HLA-B7 Tg mice were vaccinated with 100 micrograms of individual hTRT peptide together with 120 micrograms of HBV helper peptide in IFA. Ten days after immunization, spleen lymphocytes were restimulated in vitro with peptide and fresh, irradiated syngeneic APC. Restimulations were performed on a weekly basis. A standard 4 hour ⁵¹Cr-release assay was performed on day 5 after in vitro restimulation, using RMA-B7 cells pulsed with the homologous hTRT peptide as targets and an E:T ratio of 25:1. Results are expressed as the mean specific lysis plus or minus standard deviation of responder mice only, whose number is indicated in each panel. Tests were run in duplicate.

FIG. 2. Examples of CTL responses induced in vivo by immunization with p277 and p1123. Spleen lymphocytes of HLA-B7 Tg immunized mice were restimulated in vitro with the homologous hTRT peptide on a weekly basis. A standard 4 hour ⁵¹Cr-release assay was performed, using RMA-B7 cells pulsed or not pulsed with peptide as targets, at the indicated E:T ratios. CTL assay was performed after one (a and b), two (c and d) and three (e and f) rounds of in vitro restimulation.

FIG. 3. Examples of CTL induction in a small scale in vitro immunization assay using normal donor PBMC. HLA-B7⁺ human PBMC were immunized in vitro in a 96 well plate assay, and tested for specific lysis of T2-B7 pulsed with peptide at day 10-11. The micro-CTL assay was performed as described in Material and Methods. All cultures but those with p444 were set with PBMC from the same donor.

FIG. 4. Characterization of human CTL generated by in vitro immunization. An example of one of two HLA-B7⁺ normal donor PBMC from and a prostate cancer patient. Immunization in vitro was performed using a conventional method (12). (A) Specific lysis of T2-B7 cells pulsed with p1123 by CTL generated in normal donor PBMC. CTL were tested after 5 cycles of in vitro restimulation with homologous peptide. (B) Surface phenotype analysis using anti-CD3 and anti-CD8 monoclonal antibodies of the CTL shown in panel A. The percentage of double positive cells is indicated. (C) Specific lysis of T2-B7 cells pulsed with p1123 by CTL generated in prostate cancer patient PBMC. CTL were tested after 7 cycles of in vitro restimulation with homologous peptide. (D) Surface phenotype analysis using anti-CD3 and anti-CD8 monoclonal antibodies of the CTL from the same patient shown on panel C after 5 cycles of in vitro restimulation with homologous peptide. Experiments shown in A and B are representative of set of similar data from two normal donors examined at different times.

FIG. 5. p1123 is endogenously processed in JY lymphoblastoid cells. CTL from an HLA-B7⁺ human normal donor PBMC were tested in a 4 hour ⁵¹Cr-release assay of T2-B7 cells pulsed with p1123 (A), or JY cells (B). Tests were run in duplicates at the indicated E:T ratios. CTL were used after 4 cycles of in vitro restimulation with homologous peptide. Tests were done in duplicate.

FIG. 6. The nucleic acid sequence (SEQ ID NO:1) of hTRT is shown.

FIG. 7. The amino acid sequence (SEQ ID NO:2) of hTRT is shown.

FIG. 8. Murine CTL (mCTL) specific for p1123 recognizes hTRT+ human target cells (T1-B7 and BC1-B7). A mCTL line was expanded from p1123-immunized HLA-B7 Tg mice and re-stimulated five times in vitro. (A) Four-hour ⁵¹Cr-release assay was performed with mCTL using human T2-B7 as target cells, with or without p1123 pulsing. (B) Intracellular IFN-gamma staining of mCTL upon overnight incubation with T1-B7, BC1-B7 lymphoblastoid cells and T2-B7 pulsed with p1123 (positive control) and p464 (negative control). Tests were repeated twice with similar results.

### DEFINITIONS

To facilitate an understanding of the present invention, a number of terms and phrases are defined below:

As used herein, the terms “purified” and “isolated” refer to molecules (polynucleotides or polypeptides) or organisms that are removed or separated from their natural environment. “Substantially purified” molecules or organisms are at least 50% free, preferably at least 75% free, more preferably at least 90% and most preferably at least 95% free from other components with which they are naturally associated.

The term “wild-type” refers to a gene, gene product or organism that has the characteristics of that gene, gene product or organism when isolated from a naturally occurring source. A wild type gene or organism is that which is most frequently observed in a population and is thus arbitrarily designated the “normal” or “wild-type” form of the gene or organism.

In contrast, the terms “modified,” “mutant,” and “variant” refer to a gene, gene product or organism that displays modifications in sequence and or functional properties (i.e., altered characteristics) when compared to the wild-type gene, gene product or organism. It is noted that naturally occurring mutants can be isolated; these are identified by the fact that they have altered characteristics when compared to the wild-type gene, gene product or organism.

As used herein, the term “immune response” refers to the reactivity of a subject's immune system in response to an antigen. In mammals, this may involve antibody production, induction of cell-mediated immunity, and/or complement activation. In preferred embodiments, the term immune response encompasses but is not limited to one or more of a “cytotoxic T lymphocyte response,” a “lymphocyte proliferative response,” a “cytokine response,” and an “antibody response.”

In particularly preferred embodiments, the immune response encompasses induction of CTL that are essentially specific for cells that present hTRT epitopes in the context of HLA class I molecules (e.g., HLA-A, HLA-B and/or HLA-C). In some embodiments, the cells that present hTRT epitopes are HLA class I positive cells that express hTRT or that have been pulsed with a peptide (e.g., nine to 29 amino acids in length, preferably 9, 10, 11, 12, 13, 14 or 15 amino acids, including but not limited to the peptides disclosed herein in Tables I and VI-XIX) of a hTRT protein consisting of the sequence set forth as SEQ ID NO: 2. In particularly preferred embodiments, the cells that present hTRT epitopes are hTRT-positive human tumor cell lines (e.g., melanoma, prostate, breast, colon, lung, etc.) obtained from the American Type Culture Collection (ATCC). Expression of hTRT by tumor cells is determined using art-recognized methods such as the PCR-based TRAPEZE assay of Intergen (Purchase, N.Y.). Cellular cytotoxicity of hTRT-positive target cells is measured in a ⁵¹Cr-labeled release assay at an E:T ration of 50:1. In some embodiments, tumor cell lines are incubated with 100 units/ml interferon-gamma before the assay.

The term “T cell epitope” as used herein refers to an antigenic determinant presented by a MHC class I or class II molecule for binding to a single T cell receptor. T cell epitopes are linear epitopes comprising at least seven amino acid residues. In some embodiments of the present invention, the term T cell epitope encompasses a CTL epitope, which is an antigen fragment presented by an MHC class I molecule for binding to T cell receptor on the surface of a cytotoxic T lymphocyte (e.g., generally CD8⁺), while in other embodiments the term T cell epitope encompasses a Th epitope, which is an antigen fragment presented by an MHC class II molecule for binding to T cell receptor on the surface of a helper T cell (e.g., generally CD4⁺).

The term “specific for an epitope of interest” when made in reference to an immune response refers to an increased level of the immune response to cells presenting the epitope of interest (e.g., hTRT CTL epitope such as p277, p1123, p540, p865, etc.) as compared to the level of the immune response to cells presenting a control peptide (e.g., irrelevant antigen).

The term “vaccine” as used herein refers to an immunogenic composition administered to a subject for the purpose of inducing an immune response. This term encompasses candidate prophylactic and therapeutic cancer vaccines that have not yet been demonstrated to protect a subject from developing cancer and/or to eradicate a tumor or malignant cells in a cancer patient.

The term “adjuvant” as used herein refers to any compound that when injected together with an antigen, non-specifically enhances the immune response to that antigen. Exemplary adjuvants include but are not limited to incomplete Freunds adjuvant (IFA), aluminum-based adjuvants (e.g., AIOH, AIPO4, etc), and Montanide ISA 720.

The terms “excipient,” “carrier” and “vehicle” as used herein refer to usually inactive accessory substances into which a pharmaceutical substance (e.g., hTRT peptide) is suspended. Exemplary carriers include liquid carriers (such as water, saline, culture medium, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins).

The term “control” refers to subjects or samples that provide a basis for comparison for experimental subjects or samples. For instance, the use of control subjects or samples permits determinations to be made regarding the efficacy of experimental procedures. In some embodiments, the term “control subject” refers to animals or cells receiving a mock treatment (e.g., adjuvant alone).

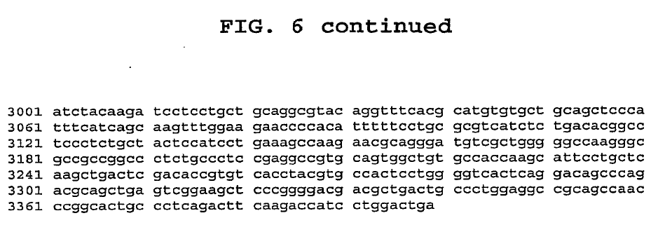

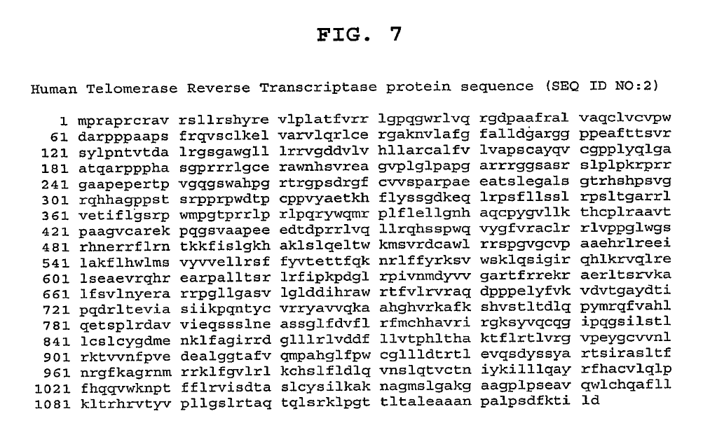

As used herein the terms “TRT,” “TERT” and “telomerase reverse transcriptase” refer to the catalytic subunit of the telomerase enzyme of eukaryotic cells that adds telomeres to the ends of chromosomes after they divide. In particular, the terms “human TRT” and “hTRT” refer to the human protein set forth in SEQ ID NO:2 (FIG. 7) encoded by the nucleic acid sequence set forth in SEQ ID NO: 1 (FIG. 6).

### DESCRIPTION OF THE INVENTION

Defining the immunogenic components of hTRT for each HLA type is a formidable task but a necessary step to develop immunotherapies to target hTRT on tumor cells in the widest assortment of the human population. Previously, this (12, 14) and other (11) laboratories identified immunogenic peptides for the most frequent HLA type, HLA-A2. The outcome of these studies was that humans possess a residual CD8 T cell repertoire for both high and low affinity hTRT peptides that can be expanded by immunization in vitro (12, 14, 16). hTRT specific CD8 T cell precursors have been reported to persist in patients with advanced cancer (12, 14, 15). Here, we expanded our systematic effort to the identification and characterization of immunogenic hTRT peptides restricted to HLA-B7. The results of the present study lead to a series of general considerations.

The conventional algorithms used here proved to be overall poor predictors of immunogenic hTRT peptides for the HLA-B7 type. Previously, we successfully used BIMAS as a way to predict and select HLA-A2 restricted hTRT peptides that fulfill desired criteria for immunogenicity similar to those studied here. In contrast, BIMAS could not predict HLA-B7 immunogenic peptides overall. For instance, p444, the top peptide according to BIMAS, was not immunogenic in vivo in HLA-B7 Tg mice, was poorly immunogenic in vitro for human PBMC, and was apparently not processed in HLA-B7 Tg mice immunized with full length hTRT pDNA. Not surprisingly, p444 actual binding avidity for the HLA-B7 molecule was also poor, hence pointing to a discrepancy between predicted affinity, actual avidity and immunogenic function. SYFPEITHI did not predict two peptides (p966 and p464), which were poorly immunogenic, but at the same time did not distinguish between immunogenic and non-immunogenic peptides among the remaining five peptides studied. Finally, predictions based on proteasome cleavage were found not to be useful. For instance, the two peptides with the highest predicted probability for processing and immunogenicity turned out to be non-immunogenic in one case (p966) and poorly immunogenic in the other case (p342). This algorithm did, however, predict p277. Collectively, none of the three algorithms used to guide the initial selection of peptides was per se able to discriminate peptides that fulfill prerequisites for immunogenicity.

In vitro immunization studies support the conclusion that there exists a residual CD8 T cell repertoire for the majority (5 out of 7) peptide specificities investigated. Since these peptides also possess good binding avidity for the HLA-B7 molecule, the present findings indicate that thymic negative selection (central tolerance) of hTRT CD8 T cell clonotypes restricted to HLA-B7 did not occur or occurred to a only limited extent. The response of HLA-B7 Tg mice to in vivo immunization with peptide in immunological adjuvant was immediate and stronger than that of HLA-A2 Tg mice similarly immunized (12, 14, 39). It appears as if, at least with respect of hTRT, HLA-B7/peptide complexes are highly immunogenic. Similarly, high immunogenicity was documented in studies where HLA-B7 Tg mice were immunized with influenza virus peptides (28).

The supertype binding studies proved to be an excellent final checkpoint in the selection of immunogenic peptides. For instance, p1123 and to a lesser degree p277 and p1107, bound to various alleles of the HLA-B7 supertype. Taken together the results of our study indicate that candidate immunogenic peptides need to satisfy at least two general criteria; good avidity interaction with the HLA-B7 molecule and good supertype binding. One may also need to consider the quality of the interaction between the MHC/peptide complex with the TCR as an additional factor in immunogenicity. As to the second characteristic, our data indicate that supertype binding peptides are preferentially processed and possess a selective advantage for interaction with molecules of the transporter associated with antigen processing (TAP) complex (14, 44, 45). Nonetheless, an understanding of the mechanism(s) is not necessary in order to make and use the present invention, and it is not intended that the present invention be limited to any particular mechanism.

In conclusion, we presented the successful identification of several immunogenic hTRT peptides restricted to HLA-B7. We show that this identification required a multi-step approach and involved an ensemble of in vitro and in vivo steps using both mice and human PBMC. This implies that the selection of immunogenic peptides for potential clinical use rests on a series of checkpoints and an element of empiricism overall. To date, such systematic approach has enabled the identification of HLA-A2 (10), and now HLA-B7 peptides with characteristics of immunogenicity that could justify their use in immunotherapy of cancer patients. Together, HLA-A2 and HLA-B7 account for ˜60% of the Caucasian population. If one takes into account supertype binding of some of the peptides identified in this study one may achieve greater than 70% coverage irrespective of ethnicity. Thus, for a complete coverage of the human population, immunogenic peptides for the alleles accounting for the remaining 30-40% of the population still need to be identified systematically using a strategy similar to the one followed herein.

### Experimental

The following example is provided in order to demonstrate and further illustrate certain preferred embodiments and aspects of the present invention and is not to be construed as limiting the scope thereof. In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); fig (micrograms); ng (nanograms); l or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); PCR (polymerase chain reaction); TRT (telomerase reverse transcriptase); WT (wild type); Tg (transgenic); TCR (T cell receptor); Th (helper T cell); MHC (major histocompatibility complex); mAb (monoclonal antibody), APC (antigen presenting cell); and CTL (cytotoxic T lymphocyte).

### Materials and Methods

Mice. HLA-B7 transgenic mice express a chimeric HLA-B7/H2-D^{b} MHC Class I molecule, are on a C57BL/6 background and have been previously described (28). Mice were originally produced at the Institut Pasteur (Paris, France). A colony was bred and maintained under specific pathogen-free conditions in the vivarium of the University of California, San Diego (La Jolla, Calif.). All experimental procedures were performed according to an approved protocol and the National Institute of Health Guide for the Care and Use of Laboratory Animals.

Cell lines. The human T2-B7 transfectants and murine RMA-B7 transfectants lines have been transfected with the HLA-B*0702 allele as described previously in (28, 29). The Epstein Barr Virus transformed B lymphoblastoid (HLA-A2/B7) JY cells were obtained from Dr. Antonella Vitiello (PRI Johnson & Johnson, La Jolla, Calif.).

Human blood cells. Buffy coats from HLA-B7⁺ normal donors were purchased from the San Diego Blood Bank (San Diego, Calif.). Prostate cancer patients were recruited through the Division of Hematology Oncology and blood was obtained by venipuncture. HLA-B7 positivity was assessed by flow cytometry. Experiments were performed in accordance with approved Institutional Review Board (IRB) protocols.

Peptides and monoclonal antibodies. All synthetic peptides were purchased from the Peptide Synthesis Core Facility of Ohio State University (Columbus, Ohio). The monoclonal antibody against HLA-B7, BB7.1, was purchased from American Tissue Type Collection (Manassas, Va.). Other antibodies used were fluorescein isothiocyanate (FITC)-conjugated mouse IgG anti-human CD8 Beta (mAb 53-6.7) and phycoerythrin (PE)-conjugated mouse IgG anti-human CD3 (BD PharMingen, San Diego, Calif.), and FITC-conjugated goat anti-mouse IgG antibody (Jackson Immunoresearch, West Grove, Pa.).

Predictive algorithms. The following predictive algorithms were used: (A) BIMAS algorithm, which is based on highly favorable and unfavorable dominant anchor residues, as well as auxiliary anchor residues, and sores peptides according to a coefficient (30) (access via: thr.cit.nih.gov/molbio/hla_bind/). (B) SYFPEITHI algorithm, which is based known T cell epitopes and MHC ligands (31, 32) (access via: www.uni-tuebingen.de/uni/kxi/) and takes into consideration the amino acids in the anchor and auxiliary anchor positions, and scores peptides according to the cumulative (positive or negative) effects of contributing amino acids with ideal anchor residues accounting for 10 points and amino acids regarded as having a negative effect on binding accounting for −1 and 3 points. (C) PAProC (Prediction Database for Proteasomal Cleavages) algorithm, which is a computer-based theoretical model for the cleavage of substrate proteins by yeast and human 20S proteasomes. PAProC predicts cleavability of amino acids sequence (cuts per amino acids) and individual cleavages (positions and estimated strength). Specifically, we used the Type III model, based on human erythrocyte proteasome cleavage of enolase and ovalbumin (33, 34) (access via: www.paproc.de/).

MHC binding assays. Relative avidity measurements. The relative avidity of hTRT peptides for HLA B7 was measured using a MHC stabilization assay on T2-B7 cells in comparison with a reference peptide as described previously (14). Results are expressed as values of relative avidity, which is the ratio of the concentration of test peptide necessary to reach 20% of the maximal binding by the reference peptide, so that the lower the value the stronger the binding.

Supertype analysis. Quantitative assays to measure the binding affinity of peptides to purified HLA B7-supertype molecules (B*0702, B*3501, B*5101, B*5301, B*5401) and B*0801 were based on the inhibition of binding of a radiolabeled standard peptide, and were performed as previously described (22, 35). Briefly, 1-10 nM of radiolabeled peptide was co-incubated at room temperature with 1 microM to 1 nM of purified MHC in the presence of 1-3 μM human beta₂-microglobulin (Scripps Laboratories, San Diego, Calif.) and a cocktail of protease inhibitors. After a two-day incubation, binding of the radiolabeled peptide to the corresponding MHC class I molecule was determined by capturing MHC/peptide complexes on Greiner Lumitrac 600 microplates (Greiner Bio-one, Longwood, Fla.) coated with the W6/32 antibody, and measuring bound counts per minute (cpm) using the TopCount microscintillation counter (Packard Instrument Co.). Results are expressed as the concentration of peptide yielding 50% inhibition of the binding of the radiolabeled reference peptide. Peptides were typically tested at 6 different concentrations covering a 100,000-fold dose range, and in 3 or more independent assays. Under the conditions utilized, where [label]<[MHC] and IC₅₀≧[MHC], the measured IC₅₀ values are reasonable approximations of the true K_{d} values.

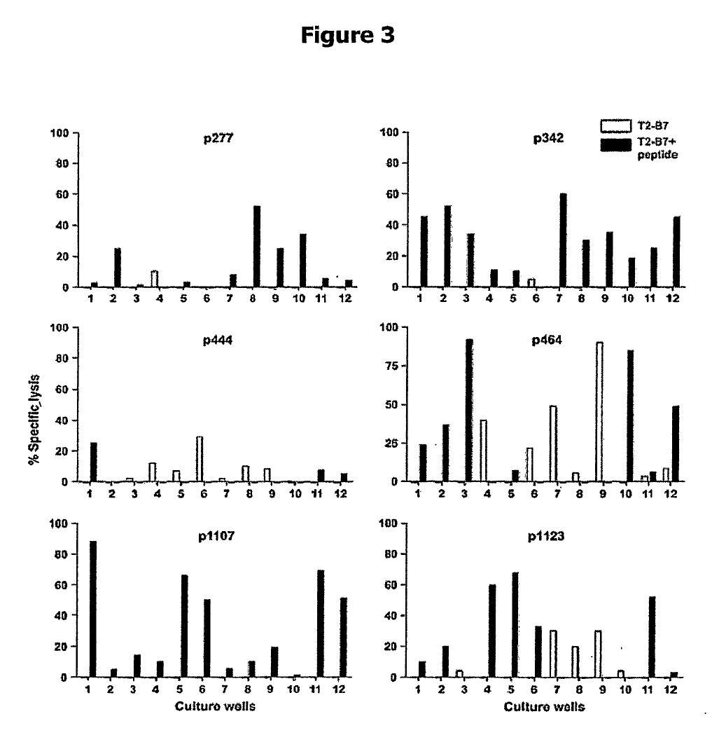

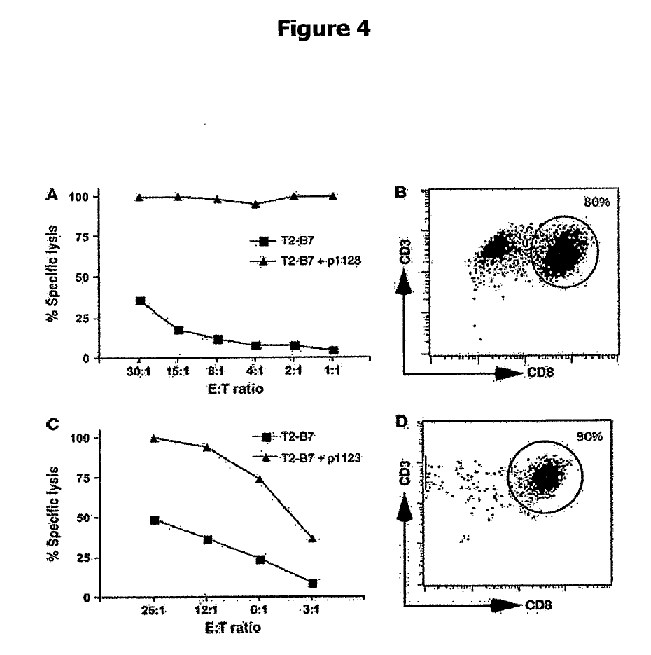

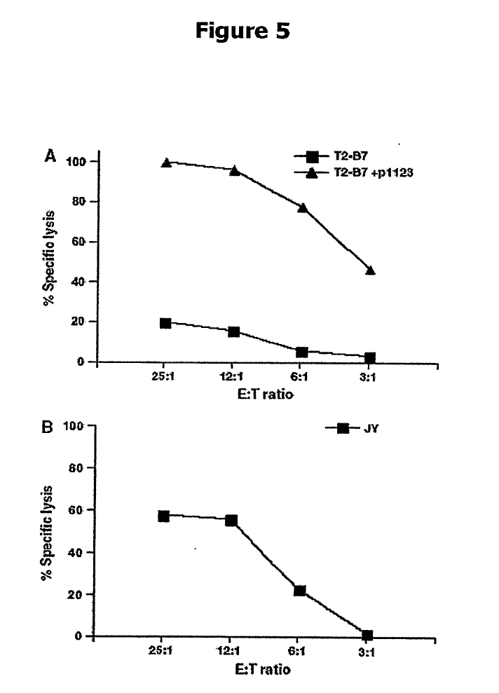

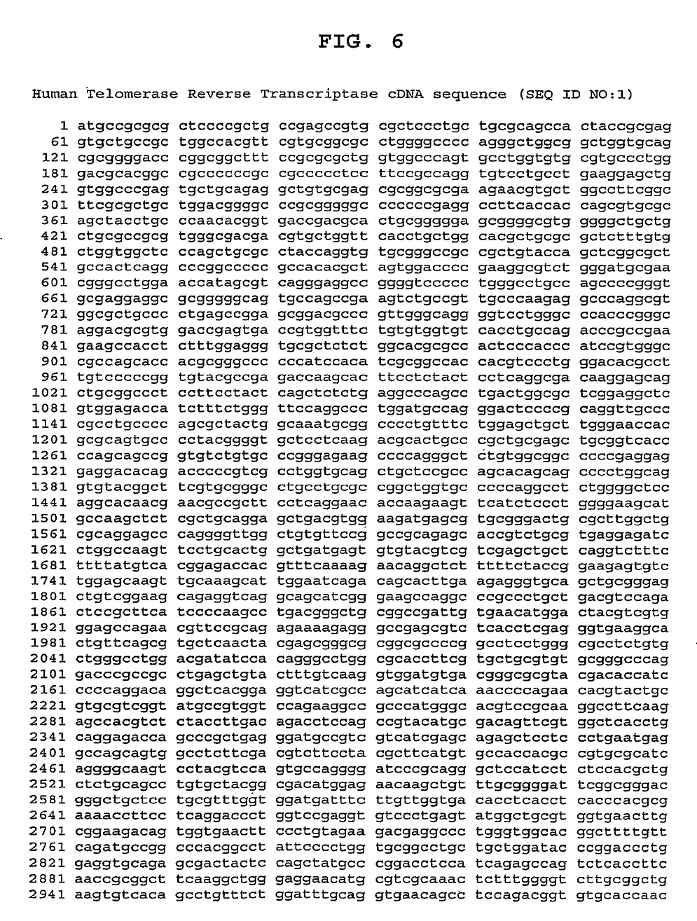

In vitro immunization procedures. In experiments shown in Table III and FIG. 3, immunizations were performed in 96 well plates. Briefly, 2×10⁵ irradiated (6000 rads) human PBMC were plated in 96 (flat) well-plate in 100 micro-liters of complete human medium (RPMI 1640 medium containing 10% heat inactivated human AB serum, 2 mM glutamine, 50 micro-grams/ml streptomycin and 50 micro-grams/ml penicillin) with 100 micro-grams/ml of peptide. 12 wells per peptide were plated per patient. Then 2×10⁵ PBMC in 100 micro-liters of complete human medium were added into each well. Four days later 100 micro-liters of medium were replaced with 100 micro-liters of fresh complete human medium containing 80 IU/ml of IL-2. At day 6-7, 100 IU/ml of IL-2 were added and wells were split into two. On day 10-11 micro-cytotoxicity assay was performed. In experiments shown in FIGS. 4 and 5 human PBMC were stimulated in vitro in 24 well plate with autologous, irradiated, peptide-pulsed adherent cells in the presence of IL-2 and IL-7 as previously described (12). On day 4 to 5 after restimulation, effector CTL were tested in a standard ⁵¹Cr-release assay.

In vivo immunization procedures. Peptide immunization. HLA B7 transgenic mice (28) were injected s.c. at the base of the tail with 100 micro-grams of hTRT peptides along with 120 micro-grams of I-A^{b} MHC Class II helper peptide 128-140 of the hepatitis B virus core protein in incomplete Freunds' adjuvant as described previously (12). Long-term CTL lines were maintained in culture by weekly restimulation with irradiated, peptide-pulsed syngeneic spleen cells in RPMI-1640 medium containing 10% heat inactivated fetal bovine serum, 2 mM glutamine, 5×10⁻⁵ M 2-Mercaptoethanol, 50 micro-grams/ml streptomycin, and 50 micro-grams/ml penicillin (complete medium) and supplemented with 40 IU/ml of recombinant human IL-2.

DNA immunization. A DNA vector coding for the hTRT expressed under the control of CMV promoter was purified on plasmid Giga Kit columns under endotoxin-free conditions (Qiagen, Hilden, Germany). Anesthetized HLA-B*0702 transgenic mice were injected with 50 micro-liters of cardiotoxin into each tibialis anterior muscle 5-6 days prior DNA injection. For vaccination, 50 micro-liters of DNA (1 micro-gram/micro-liter in PBS) was injected into each pretreated muscle at day 0 and day 14. Ten days later, spleen cells of individual mice were separately restimulated in vitro with each relevant peptide (10 micrograms/ml) for 6 days. Effector CTL cells were tested in a standard 4 hr ⁵¹Cr-release assay, using RMA-B7 cells (HLA-B*0702 transfected RMA cells) pulsed with test peptide or control peptide (CMV p65-derived R10TV restricted to HLA-B7). Specific % lysis as indicated below. In vivo immunization procedures were preformed in accordance with approved animal protocols at the University of California, San Diego or the Pasteur Institute, respectively.

CTL assays. Both murine and human CTL were detected by the ⁵¹Cr release assay performed as previously described (14). Briefly, HLA-B7⁺ antigen presenting cells (RMA-B7 or T2-B7 cells) were labeled for 1 hr with 100 micro-Ci of Na₂⁵¹CrO₄ (Perkin Elmer). Washed cells (5×10³ per well) were mixed in 96-well plates in 100 micro-liters/well with each peptide (at 10 micro-grams/ml or lower concentration) and 100 micro-liters of the CTL effector cells (at various E:T ratio) in RPMI medium. The plates were incubated for 4-5 hrs at 37° C. (5% CO₂). The supernatants were harvested and counted on a Wallac 1470 Wizard Gamma counter. The percent lysis was calculated as 100 (cpmₑₓₚ−cpmₛₚₒₙₜ)/(cpmₘₐₓ−cpmₛₚₒₙₜ).

FACS analysis. The phenotypic characteristics of in vitro expanded CTL were determined by FACS analysis. Briefly, on day 6 or 7 after stimulation, cells (0.5×10⁶) were incubated with FITC-conjugated mouse anti-human CD8 mAb and PE-conjugated mouse anti-human CD3 mAb (2 micro-grams/ml) in Hank's Balanced Solution containing 0.1% BSA and 0.05% sodium azide for 30 min at 4° C. For human PBMC typing, cells were incubated with 10 micro-1 of BB7.1 mouse B cell hybridoma supernatant for 20 min at 4° C., followed by 30 min incubation with FITC-conjugated rabbit anti-mouse IgG antibody. Samples were analyzed on a FACSCalibur (Becton Dickinson, San Jose, Calif.). One hundred thousand events were collected and analyzed using the CellQuest software (Becton Dickinson).

### Results

### Selection of Peptides on Predicted Algorithms.

To limit the number of candidate peptides to a manageable panel we used two predictive algorithms BIMAS and SYFPEITHI. These were used independently to predict nine aminoacid peptides for the HLA-B*0702 allele which accounts for the majority of the members of the HLA-B7 type (27). While BIMAS predicts HLA binding based on overall binding characteristics and the presence of canonic anchor residues, SYFPEITHI predicts peptides whose binding characteristics are extrapolated from naturally occurring MHC ligands as a matrix database. PAProC (Prediction Database for Proteasomal Cleavages), which predicts the proteasomal cleavage of full-length proteins, was used to define cleavage accessibility.

We initially selected ten 9mer peptides with high predicted scores in either of the two algorithms or both, and synthesized seven peptides (Table I). These peptides were selected based on a consensus prediction by both BIMAS and SYFPEITHI. Among the seven peptides only three had a score greater than 180 using BIMAS, and all but two had a score of 23 using SYFPEITHI. Interestingly, the two peptides that could not be predicted using SYPEITHI scored among the best using BIMAS.

TABLE I
Prediction of HLA-B7 binding for hTRT peptides

| | |
|---|---|
| hTRT peptides | Predictive Algorythm |

| | | | | | |
|---|---|---|---|---|---|
| | | SEQ ID | | | |
| a.a. | Sequence | NO | BIMAS | SYFPEITHI | PAProC |

| | | | | | |
|---|---|---|---|---|---|
| p277 | RPAEEATSL | 3 | 80 | 23 | XX |
| p342 | RPSFLLSSL | 4 | 80 | 23 | XXX |
| p444 | DPRRLVQLL | 5 | 800 | 23 | X |
| p464 | FVRACLRRL | 6 | 200 | NP | 0 |
| p966 | AGRNMRRKL | 7 | 180 | NP | XXX |
| p1107 | LPGTTLTAL | 8 | 80 | 23 | 0 |
| p1123 | LPSDFKTIL | 9 | 80 | 23 | 0 |

HLA-B*0702 binding affinity was predicted by BIMAS and SYFPEITHI, where for the former the minimum numerical value for 9mer peptides possessing canonical anchor residues is 180, and for the latter is 20. C-terminus proteasomal cleavage of the predicted 9mers out of the full-length (1132 amino acids) hTRT by proteasomal cleavage (PAProC). The predicted proteasomal cleavage strength is arbitrarily scored as 0 (for no cleavage), X, XX and XXX (for cleavage strength).

NP=not predicted

Next, we assessed the actual binding avidity for HLA-B7 (HLA-B*0702). Two independent assays were used: binding stabilization assay on T2-B7 cells by flow cytometry (12) and a competitive solid-phase radioimmunoassay on immobilized purified HLA-B7 molecule (35). As shown in Table II five out of seven peptides (p277, p342, p464, p1107 and p1123) displayed high avidity binding. The two peptides with weak binding (p444 and p966) were among the top three peptides predicted by BIMAS. There was excellent concordance between the two types of binding assays utilized.

TABLE II
Relative avidity of predicted hTRT peptides for HLA-B7

| hTRT | | IC50^{b} |
|---|---|---|
| peptide | RA^{a} | (nM) |
| p277 | 4.7 | 6.3 |
| p342 | 2.5 | 0.56 |
| p444 | >20 | 239 |
| p464 | 3.2 | 4.1 |
| p966 | >20 | — |
| p1107 | 3.8 | 0.96 |
| p1123 | 1.8 | 11 |

^{a}Relative avidity was tested by MHC stabilization assay on T2-B7.
^{b}IC50 was calculated by competition solid-phase radioimmunoassay.
Dash indicates an IC50 > 50,000 nM

### In Vivo Immunization of HLA-B7 Tg Mice

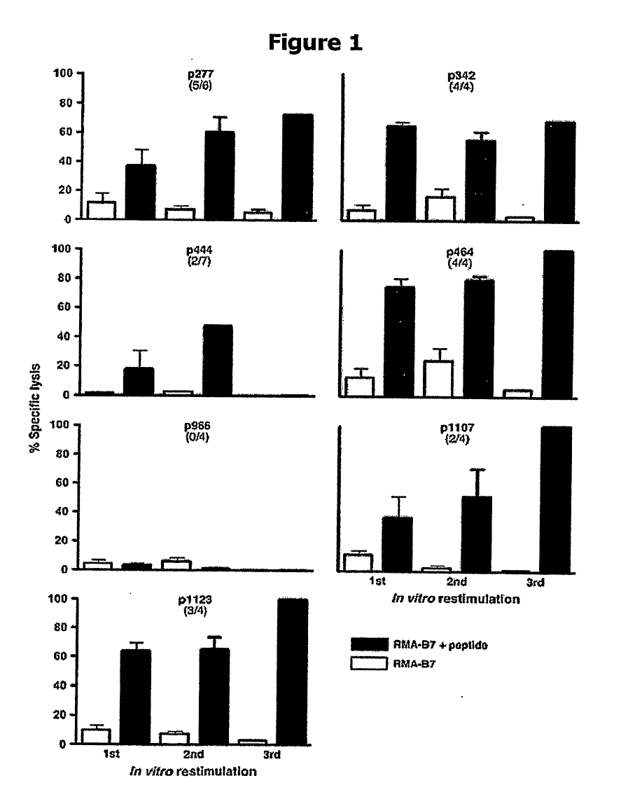

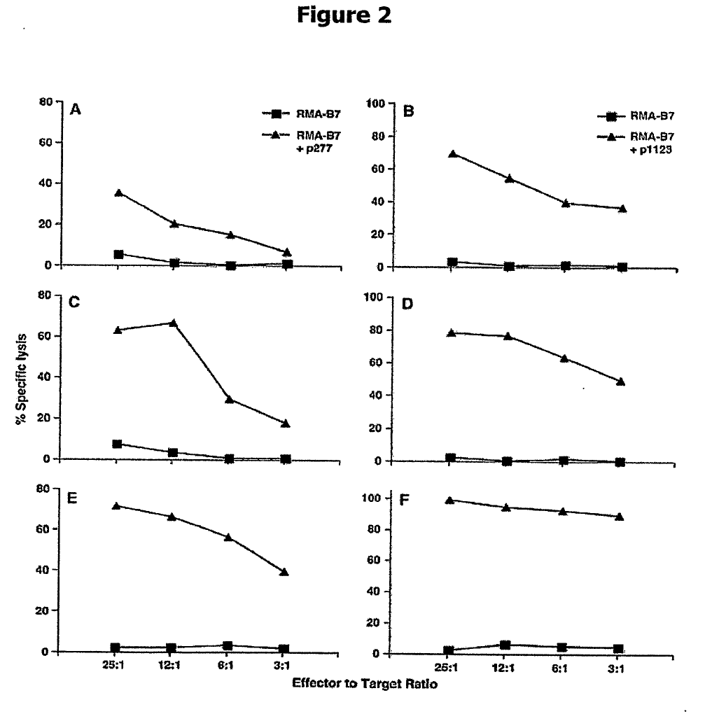

In order to assign immunogenicity to each of the peptides and correlate this property with the binding characteristics and the scores of the predictive algorithms, we immunized HLA-B7 Tg mice (28). Ten to eleven days after immunization mice were sacrificed, the spleen harvested and splenocytes put in culture with LPS/Dextran activated APC, and tested in a 4 hour ⁵¹Cr-release assay. As shown, only five out of seven peptides yielded a meaningful, specific CTL response even after a third cycle of in vitro restimulation (FIG. 1). All immunogenic peptides induced a response from the first in vitro restimulation and this response increased upon subsequent rounds of antigen restimulation. An example of CTL for two of the immunogenic peptides is shown in FIG. 2. As noted the lysis of peptide-pulsed RMA-B7 target cells increased at each round of in vitro restimulation. No lysis occurred on RMA-B7 cells not pulsed with peptide. Thus, the in vivo results together with the actual measure of the avidity of HLA-B7 binding avidity distinguished two groups of 9mer hTRT peptides. One group (p277, p342, p464, p1107 and p1123), displayed both high binding in vitro and good immunogenicity in vivo. The other group (p444 and p966), showed poor binding and poor immunogenicity.

In Vitro Immunization of Human PBMC from Normal Donors

To further assess the immunogenicity of the selected peptide candidates as well as their ability to expand precursor CD8 T cell in human PBMC, the following experiment was performed. PBMC from eight HLA-B7⁺ normal donors were screened in a small scale in vitro immunization assay (96 well plate assay) to determine the level of responses against each individual peptide. The cumulative data of this screening step are shown in Table III. As indicated the response to these peptides varied among the eight donors. Overall, two peptides (p277 and p1123) yielded strong responses in the majority of the subjects. Three peptides p342, p464 and p107) induced strong responses but in fewer instances only. Notably, p444 that was poorly immunogenic in vivo in HLA-B7 Tg mice also displayed poor immunogenicity in this micro-CTL assay. The response against p966 was not tested because of repeated negative results in HLA-B7 Tg mice. Thus, the results of this in vitro assay narrowed the spectrum of immunogenic peptides beyond those identified in vivo in HLA-B7 Tg mice. A typical result of this type of analysis is shown in FIG. 3, which depicts the induction of CTL and their specificity in each of the twelve wells. As shown, there is considerable variability in the number of positive wells per peptide as well as in the percentage lysis which itself varied from peptide to peptide. This variation in the response to each peptide may be related to either an intrinsic characteristic of the peptide (e.g., its avidity) or a variation in the frequency of CD8 T cell precursors for that peptide among donors particularly in view of the format of the assay used.

TABLE III
CTL response in vitro following immunization of normal
donors PBMC with HLA-B7 restricted hTRT peptides

| hTRT | Donor | Donor | Donor | Donor | Donor | Donor | Donor | Donor | High | Low |
|---|---|---|---|---|---|---|---|---|---|---|
| peptide | 1 | 2 | 3 | 4 | 5 | 6 | 7 | 8 | R/Total | R/Total |
| p277 | >50% | >50% | >50% | >50% | >50% | <25% | <25% | <25% | 5/8 | 0/8 |
| p342 | >25% | >25% | >25% | >50% | 0 | <25% | >25% | 0 | 1/8 | 4/8 |
| p444 | ND | ND | <25% | >25% | 0 | 0 | 0 | <25% | 0/8 | 1/8 |
| p464 | >50% | >50% | 0 | >25% | <25% | <25% | <25% | 0 | 2/8 | 1/8 |
| p966 | ND | ND | ND | ND | ND | ND | ND | ND | ND | ND |
| p1107 | >50% | >50% | 0 | >50% | >25% | <25% | >25% | 0 | 3/8 | 2/8 |
| p1123 | >50% | >50% | >50% | <25% | >50% | >50% | >50% | >50% | 7/8 | 0/8 |

PBMC from HLA-B7⁺ normal blood donors were pulsed with the candidate peptide in 96 well plate assay (described in Material and Methods), and tested for lysis of T2-B7 pulsed with peptide on day 10-11. A micro ⁵¹Cr-release assay was performed as described in Material and Methods. Responders were considered at >50% specific CTL lysis. CTL assays were performed at an approximate E:T ratio of 10:1. ND not done

### In Vivo Processing

Next, we established which among the various candidate peptides was processed and presented from full-length hTRT. To this end, we immunized HLA-B7 Tg mice with hTRT plasmid DNA. Mice were sacrificed on day 24, and splenocytes were restimulated in vitro with each of the following peptides: p277, p342, p444, p464, p1107 and p1123. As shown in Table IV some but not all the peptides were processed and presented in vivo. Three peptides (p277, p1107 and p1123) yielded greater CTL responses than the other peptides, implying either preferential processing and/or better immunogenicity once displayed at the surface of the APC. The remaining three peptides (p342, p444, p464) were marginally immunogenic if any. Based on this analysis, it appears that only three of the original seven peptides were processed and presented efficiently in vivo. Interestingly, we found that among the three most immunogenic peptides only one (p277) was predicted by PAProC, whereas the other two (p 1107 and p1123) were not (Table I). Thus, selection using PAProC algorithm was per se unable to predict hTRT peptides that would be cleaved and become immunogenic in vivo.

TABLE IV
In vivo processing and immunogenicity
of hTRT peptides in HLA-B7 Tg mice

| hTRT | | | |
|---|---|---|---|
| peptide | Responders/Total | % Responders | Specific Lysis (%) |
| p277 | 4/7 | 57 | 6, 3, 20, 16, 34, 6, 31 |
| p342 | 2/7 | 29 | 3, 4, 7, 13, 6, 2, 20 |
| p444 | 0/4 | 0 | 5, 8, 6, 4 |
| p464 | 2/6 | 17 | 9, 16, 12, 9, 7, 2 |
| p1107 | 3/6 | 50 | 19, 8, 30, 20, 9, 4 |
| p1123 | 3/6 | 50 | 20, 9, 14, 31, 11, 9 |

HLA-B7 Tg mice were immunized with a pDNA coding for full-length hTRT under the CMV promoter. ⁵¹Cr-release assay was performed after 6 days of in vitro restimulation with respective peptide. Mice were considered responders when >10% specific lysis was observed. Tests were run in duplicate at an E:T ratio of 60:1, using RMA-B7 target cells.

### Supertype Analysis

HLA molecules are highly polymorphic posing problems to the identification of peptides, which could be used to cover the totality of the human population. However, HLA alleles can be clustered into a relatively small number of groups termed supertypes (21). The HLA-B7 supertype includes ten alleles (22). Here, we decided to test the selected hTRT peptides for their ability to bind five out of ten members of the HLA-B7 supertype (B*3501, B*5101, B*5301, B*5401) and B*0801. The B*0801 allele shares binding features with B*0702, although is not officially part of the HLA-B7 supertype. This analysis had the purpose to further narrow the selection of putative HLA-B7 immunogens based on supertype binding (Table V). Only one peptide (p1123) had measurable avidity for all alleles examined. Another peptide (p1107) bound with high avidity three out of five alleles. Two additional peptides (p277 and p342) bound four alleles with intermediate avidity. The remaining peptides (p444, p464 and p966) displayed little HLA-B7 supertype binding. Thus, it appears as if the peptides retained through the in vitro and in vivo screening processes described above for immunogenicity and processing in vivo, ranked best as HLA-B7 supertype binders. This demonstrates that the supertype analysis is a pivotal step in refining the selection process.

TABLE V
HLA-B7 supertype binding assay

SEQ

| hTRT | | ID | HLA class I binding capacity (IC50 nM) | | | | | |
|---|---|---|---|---|---|---|---|---|
| peptide | Sequence | NO | B*0702 | B*3501 | B*5101 | B*5301 | B*54011 | B*0801 |

| | | | | | | | | |
|---|---|---|---|---|---|---|---|---|
| p277 | RPAEEATSL | 3 | 6.3 | 510 | — | 10618 | 45158 | 207 |
| p342 | RPSFLLSSL | 4 | 0.56 | 1019 | — | 2199 | 12648 | 37 |
| p444 | DPRRLVQLL | 5 | 239 | — | 7069 | — | 21933 | 217 |
| p464 | FVRACLRRL | 6 | 4.1 | — | — | — | 18843 | 123 |
| p966 | AGRNMRRKL | 7 | — | — | — | — | 34065 | — |
| p1107 | LPGTTLTAL | 8 | 0.96 | 132 | — | 120 | — | 192 |
| p1123 | LPSDFKTIL | 9 | 11 | 5 | 1625 | 2.4 | 19877 | 74 |

Dash indicates an IC50 > 50000 nM

Characterization of Human CTL Against p1123

To better characterize the response against the peptide with the best characteristics for immunogenicity overall (p1123), new in vitro immunization experiments were performed, using PBMC from two HLA-B7⁺ normal blood donors and one cancer patient. These experiments were performed using a conventional in vitro immunization assay (12). After repeated rounds of in vitro restimulation high efficiency CTL were induced that specifically killed T2-B7 target cells pulsed with p1123 (FIG. 4A). These CTL showed to CD3/CD8 double positive T cells (80%) (FIG. 4B). Thus, p1123 expanded CD8 T cell precursors, which developed into CTL. A similar approach was used with PBMC from a prostate cancer patient. Again, after repeated restimulations we were able to expand CTL that killed T2-B7 target cells pulsed with p1123 (FIG. 4C). Compared with the efficiency of induction observed in both normal blood donors, the CTL induced in the cancer patient were less efficient. The activity was seemingly mediated by CD3/CD8⁺ lymphocytes double positive T cells (75%) as indicated by FACS analysis (FIG. 4D). Collectively, these data confirm that CD8 T cell precursors for p1123 exist in the normal CD8 T cell repertoire, and persist after cancer development.

Finally, it was important to demonstrate that CTL against p1123 were able to lyse transporter associated with antigen processing protein (TAP) competent/hTRT positive target cells. To this end, we used the JY (a HLA-A2⁺/B7⁺ EBV transformed B lymphoblastoid human cell line), which is highly positive for hTRT (our unpublished data). CTL from normal donors that efficiently killed T2-R7 target cells pulsed with p1123, also killed JY cells in the absence of any peptide pulsing (FIG. 5), suggesting that p1123 is naturally processed from endogenous hTRT, and that HLA-B7/p1123 complexes are presented at the cell surface in a way that is recognized by CTL induced by peptide immunization.

A mCTL Line Recognizes Human Cells

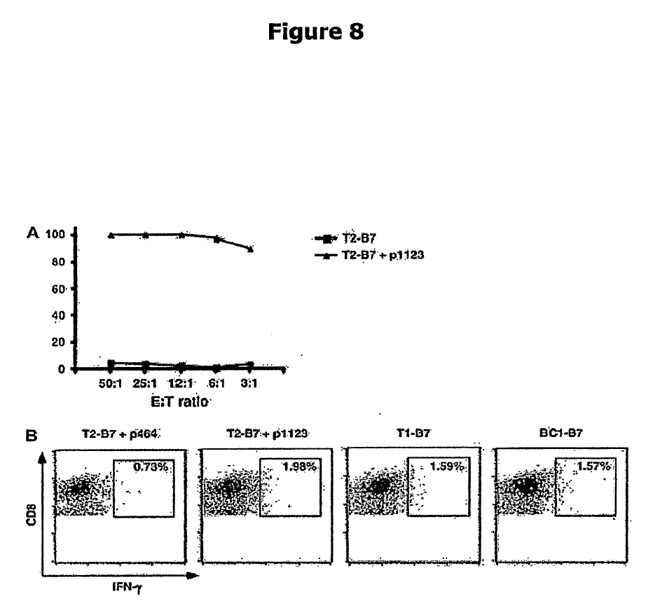

To further characterize the endogenous processing and presentation of p1123 in human cells, we used a mCTL line specific for p 1123 with high lytic activity for human target cells (T2-B7) pulsed with peptide (p1123) (FIG. 8A). Two HLA-B7+ human lymphoblastoid cells were used, T1-B7 and BC1-B7. Although TAP-deficient T2-B7 cells pulsed with p1123 are highly susceptible to lysis by mCTL, non-pulsed TAP competent hTRT+HLA-B7+EBV-transformed B lymphoblastoid human cell lines, T1-B7 and BC1-B7, were not. This indicates that the low-affinity interaction between the murine CD8 coreceptor molecule and the human MHC may be compensated by the abundance of MHC-peptide complexes on T2-B7 cells pulsed with peptide.

As an alternative approach, we tested intracellular synthesis IFN-gamma in a mCTL line specific for p1123 in the presence of T1-B7 and BC1-B7 cells, reasoning that specific recognition of p1123 would engender IFN-gamma synthesis. This was assessed by measuring intracellular staining. As shown in FIG. 8B, overnight contact with T1-B7 and BC1-B7 lymphoblastoid cells produced an increase in CD8/IFN-gamma double-positive cells. This was only slightly at variance with the percentage CD8/IFN-gamma double positive CTL incubated with control T2-B7 cells pulsed with p1123 (positive control). This confirms, therefore, endogenous processing and presentation of hTRT p 1123 in human cells.

All publications and patents mentioned in the above specification are herein incorporated by reference. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art, are intended to be within the scope of the present invention.

### HLA-A3 Supertype hTRT Peptides

TABLE VI
HLA-A3 hTRT peptides

| | | | |
|---|---|---|---|
| 1 | 535 | RLREEILAK | SEQ ID NO: 14 |
| 2 | 1081 | KLTRHRVTY | SEQ ID NO: 15 |
| 3 | 817 | AVRIRGKSY | SEQ ID NO: 16 |
| 4 | 740 | CVRRYAVVQ | SEQ ID NO: 17 |
| 5 | 143 | RVGDDVLVH | SEQ ID NO: 18 |
| 6 | 973 | KLFGVLRLK | SEQ ID NO: 19 |
| 7 | 130 | ALRGSGAWG | SEQ ID NO: 20 |
| 8 | 79 | ELVARVLQR | SEQ ID NO: 21 |
| 9 | 378 | RLPRLPQRY | SEQ ID NO: 22 |
| 10 | 418 | AVTPAAGVC | SEQ ID NO: 23 |

TABLE VII
HLA-A*1101 hTRT peptides

| | | | |
|---|---|---|---|
| 1 | 535 | RLREEILAK | SEQ ID NO: 24 |
| 2 | 562 | YVTETTFQK | SEQ ID NO: 25 |
| 3 | 973 | KLFGVLRLK | SEQ ID NO: 26 |
| 4 | 881 | KTFLRTLVR | SEQ ID NO: 27 |
| 5 | 550 | SVYVVELLR | SEQ ID NO: 28 |
| 6 | 83 | RVLQRLCER | SEQ ID NO: 29 |
| 7 | 995 | QTVCTNIYK | SEQ ID NO: 30 |

TABLE VIII
HLA-A*3101 hTRT peptides

| | | | |
|---|---|---|---|
| 1 | 83 | RVLQRLCER | SEQ ID NO: 31 |
| 2 | 881 | KTFLRTLVR | SEQ ID NO: 32 |
| 3 | 1003 | KILLLQAYR | SEQ ID NO: 33 |
| 4 | 550 | SVYVVELLR | SEQ ID NO: 34 |
| 5 | 513 | SVRDCAWLR | SEQ ID NO: 35 |

TABLE IX
HLA-A*6801 hTRT peptides

| | | | |
|---|---|---|---|
| 1 | 147 | DVLVHLLAR | SEQ ID NO: 36 |
| 2 | 605 | EVRQHREAR | SEQ ID NO: 37 |
| 3 | 663 | SVLNYERAR | SEQ ID NO: 38 |
| 4 | 639 | VVGARTFRR | SEQ ID NO: 39 |
| 5 | 638 | YVVGARTFR | SEQ ID NO: 40 |
| 6 | 83 | RVLQRLCER | SEQ ID NO: 41 |
| 7 | 550 | SVYVVELLR | SEQ ID NO: 42 |
| 8 | 55 | LVCVPWDAR | SEQ ID NO: 43 |
| 9 | 513 | SVRDCAWLR | SEQ ID NO: 44 |
| 10 | 1089 | YVPLLGSLR | SEQ ID NO: 45 |
| 11 | 79 | ELVARVLQR | SEQ ID NO: 46 |
| 12 | 727 | EVIASIIKP | SEQ ID NO: 47 |
| 13 | 135 | GAWGLLLRR | SEQ ID NO: 48 |
| 14 | 503 | LSLQELTWK | SEQ ID NO: 49 |
| 15 | 995 | QTVCTNIYK | SEQ ID NO: 50 |

### HLA-B44 Supertype hTRT Peptides

TABLE X
HLA-B*4403 hTRT peptides

| | | | |
|---|---|---|---|
| 1 | 911 | DEALGGTAF | SEQ ID NO: 51 |
| 2 | 554 | VELLRSFFY | SEQ ID NO: 52 |
| 3 | 19 | REVLPLATF | SEQ ID NO: 53 |
| 4 | 317 | WDTPCPPVY | SEQ ID NO: 54 |

TABLE XI
HLA-B*4402 hTRT peptides

| | | | |
|---|---|---|---|
| 1 | 440 | EEDTDPRRL | SEQ ID NO: 55 |
| 2 | 338 | KEQLRPSFL | SEQ ID NO: 56 |
| 3 | 19 | REVLPLATF | SEQ ID NO: 57 |
| 4 | 89 | CERGAKNVL | SEQ ID NO: 58 |
| 5 | 208 | REAGVPLGL | SEQ ID NO: 59 |
| 6 | 532 | AEHRLREEI | SEQ ID NO: 60 |
| 7 | 537 | REEILAKFL | SEQ ID NO: 61 |
| 8 | 554 | VELLRSFFY | SEQ ID NO: 62 |
| 9 | 892 | PEYGCVVNL | SEQ ID NO: 63 |
| 10 | 911 | DEALGGTAF | SEQ ID NO: 64 |
| 11 | 667 | YERARRPGL | SEQ ID NO: 65 |
| 12 | 1115 | LEAAANPAL | SEQ ID NO: 66 |

TABLE XII
HLA-B*60 hTRT peptides

| | | | |
|---|---|---|---|
| 1 | 208 | REAGVPLGL | SEQ ID NO: 67 |
| 2 | 1115 | LEAAANPAL | SEQ ID NO: 68 |
| 3 | 537 | REEILAKFL | SEQ ID NO: 69 |
| 4 | 440 | EEDTDPRRL | SEQ ID NO: 70 |
| 5 | 667 | YERARRPGL | SEQ ID NO: 71 |
| 6 | 338 | KEQLRPSFL | SEQ ID NO: 72 |
| 7 | 89 | CERGAKNVL | SEQ ID NO: 73 |

TABLE XIII
HLA-B*61 hTRT peptides

| | | | |
|---|---|---|---|
| 1 | 506 | QELTWKMSV | SEQ ID NO: 74 |
| 2 | 604 | AEVRQHREA | SEQ ID NO: 75 |
| 3 | 280 | EEATSLEGA | SEQ ID NO: 76 |
| 4 | 781 | QETSPLRDA | SEQ ID NO: 77 |
| 5 | 199 | CERAWNHSV | SEQ ID NO: 78 |
| 6 | 428 | REKPQGSVA | SEQ ID NO: 79 |
| 7 | 208 | REAGVPLGL | SEQ ID NO: 80 |
| 8 | 1115 | LEAAANPAL | SEQ ID NO: 81 |

### HLA-A1 Supertype hTRT Peptides

TABLE XIV
HLA-A*01 hTRT peptides

| | | | |
|---|---|---|---|
| 1 | 325 | YAETKHFLY | SEQ ID NO: 82 |
| 2 | 1036 | ISDTASLCY | SEQ ID NO: 83 |
| 3 | 442 | DTDPRRLVQ | SEQ ID NO: 84 |
| 4 | 699 | AQDPPPELYDF | SEQ ID NO: 85 |
| 5 | 766 | LTDLQPYMR | SEQ ID NO: 86 |
| 6 | 943 | QSDYSSYAR | SEQ ID NO: 87 |
| 7 | 838 | STLLCSLCY | SEQ ID NO: 88 |
| 8 | 764 | STLTDLQPY | SEQ ID NO: 89 |
| 9 | 938 | RTLEVQSDY | SEQ ID NO: 90 |
| 10 | 563 | VTETTFQKN | SEQ ID NO: 91 |
| 11 | 659 | KALFSVLNY | SEQ ID NO: 92 |
| 12 | 1081 | KLTRHRVTY | SEQ ID NO: 93 |
| 13 | 941 | EVQSDYSSY | SEQ ID NO: 94 |

TABLE XV
HLA-A*26 hTRT peptides

| | | | |
|---|---|---|---|
| 1 | 941 | EVQSDYSSY | SEQ ID NO: 95 |
| 2 | 552 | YVVELLRSF | SEQ ID NO: 96 |
| 3 | 727 | EVIASIIKP | SEQ ID NO: 97 |
| 4 | 565 | ETTFQKNRL | SEQ ID NO: 98 |
| 5 | 790 | VVIEQSSSL | SEQ ID NO: 99 |
| 6 | 362 | ETIFLGSRP | SEQ ID NO: 100 |
| 7 | 147 | DVLVHLLAR | SEQ ID NO: 101 |
| 8 | 1034 | RVISDTASL | SEQ ID NO: 102 |
| 9 | 281 | EATSLEGAL | SEQ ID NO: 103 |
| 10 | 327 | ETKHFLYSS | SEQ ID NO: 104 |

### HLA-A24 Supertype hTRT Peptides

TABLE XVI
HLA-A*24 hTRT peptides

| | | | |
|---|---|---|---|
| 1 | 1088 | TYVPLLGSL | SEQ ID NO: 105 |
| 2 | 845 | CYGDMENKL | SEQ ID NO: 106 |
| 3 | 167 | AYQVCGPPL | SEQ ID NO: 107 |
| 4 | 461 | VYGFVRACL | SEQ ID NO: 108 |
| 5 | 324 | VYAETKHFL | SEQ ID NO: 109 |
| 6 | 1009 | AYRFHACVL | SEQ ID NO: 110 |
| 7 | 385 | RYWQMRPLF | SEQ ID NO: 111 |
| 8 | 637 | DYVVGARTF | SEQ ID NO: 112 |
| 9 | 622 | RFIPKPDGL | SEQ ID NO: 113 |
| 10 | 869 | DFLLVTPHL | SEQ ID NO: 114 |
| 11 | 1011 | RFHACVLQL | SEQ ID NO: 115 |
| 12 | 486 | RFLRNTKKF | SEQ ID NO: 116 |

### HLA-B27 Supertype hTRT Peptides

TABLE XVII
HLA-B*2705 hTRT peptides

| | | | |
|---|---|---|---|
| 1 | 485 | RRFLRNTKK | SEQ ID NO: 117 |
| 2 | 358 | RRLVETIFL | SEQ ID NO: 118 |
| 3 | 858 | RRDGLLLRL | SEQ ID NO: 119 |
| 4 | 646 | RREKRAERL | SEQ ID NO: 120 |
| 5 | 649 | KRAERLTSR | SEQ ID NO: 121 |
| 6 | 222 | RRRGGSASR | SEQ ID NO: 122 |
| 7 | 377 | RRLPRLPQR | SEQ ID NO: 123 |
| 8 | 742 | RRYAVVQKA | SEQ ID NO: 124 |
| 9 | 810 | LRFMCHHAV | SEQ ID NO: 125 |
| 10 | 29 | RRLGPQGWR | SEQ ID NO: 126 |
| 11 | 971 | RRKLFGVLR | SEQ ID NO: 127 |
| 12 | 384 | QRYWQMRPL | SEQ ID NO: 128 |
| 13 | 229 | SRSLPLPKR | SEQ ID NO: 129 |
| 14 | 260 | GRTRGPSDR | SEQ ID NO: 130 |

TABLE XVIII
HLA-B*2702 hTRT peptides

| | | | |
|---|---|---|---|
| 1 | 470 | RRLVPPGLW | SEQ ID NO: 131 |
| 2 | 742 | RRYAVVQKA | SEQ ID NO: 132 |
| 3 | 978 | LRLKCHSLF | SEQ ID NO: 133 |
| 4 | 107 | ARGGPPEAF | SEQ ID NO: 134 |
| 5 | 536 | LREEILAKF | SEQ ID NO: 135 |
| 6 | 10 | VRSLLRSHY | SEQ ID NO: 136 |
| 7 | 357 | ARRLVETIF | SEQ ID NO: 137 |
| 8 | 630 | LRPIVNMDY | SEQ ID NO: 138 |
| 9 | 646 | RREKRAERL | SEQ ID NO: 139 |
| 10 | 858 | RRDGLLLRL | SEQ ID NO: 140 |
| 11 | 358 | RRLVETIFL | SEQ ID NO: 141 |

TABLE XIX
HLA-B*1510 hTRT peptides

| | | | |
|---|---|---|---|
| 1 | 608 | QHREARPAL | SEQ ID NO: 142 |
| 2 | 1084 | RHRVTYVPL | SEQ ID NO: 143 |
| 3 | 150 | VHLLARCAL | SEQ ID NO: 144 |
| 4 | 16 | SHYREVLPL | SEQ ID NO: 145 |
| 5 | 533 | EHRLREEIL | SEQ ID NO: 146 |
| 6 | 778 | AHLQETSPL | SEQ ID NO: 147 |
| 7 | 761 | SHVSTLTDL | SEQ ID NO: 148 |
| 8 | 1074 | CHQAFLLKL | SEQ ID NO: 149 |
| 9 | 189 | HASGPRRRL | SEQ ID NO: 150 |
| 10 | 751 | AHGHVRKAF | SEQ ID NO: 151 |

### REFERENCES

1. Blackburn, 1992. Annual Review of Biochemistry 61:113.
2. Kim et al., 1994. Science 266:2011.
3. Meyerson et al., 1997. Cell 90:785.
4. Bodnar et al., 1998. Science 279:349.
5. Morales et al., 1999. Nature Genetics 21:115.
6. Hahn et al., 1999. Nature 400:464.
7. Shay and Bacchetti, 1997. Eur J of Cancer 33:787.
8. Kim, 1997. Eur J of Cancer 33:781.
9. Nakamura et al., 1997. Science 277:955.
10. Zanetti et al., 2005. Springer Semin Immunopathol. 27:87.
11. Vonderheide et al., 1999. Immunity 10:673.
12. Minev et al., 2000. Proc Natl Acad Sci USA 97:4796.
13. Vonderheide et al., 2001. Clin Cancer Res 7:3343.
14. Hernandez et al., 2002. Proc Natl Acad Sci USA 99:12275.
15. Amarnath et al., 2004. Int J Oncol 25:211.
16. Vonderheide et al., 2004. Clin Cancer Res 10:828.
17. Su et al., 2003. Cancer Res 63:2127.
18. Su et al., 2005. J Immunol 174:3798.
19. Zanetti, 2003. Hum Gene Ther 14:301.
20. Yewdell and Bennink, 1992. Adv Immunol 52:1.
21. Sette and Sidney, 1999. Immunogenetics 50:201.
22. Sidney et al., 1996. J Immunol 157:3480.
23. Arai et al., 2001, Blood, 97:2903.
24. Lee, 1990. In The HLA System. J. Lee, ed. Springer-Verlag, New York, p. 141.
25. Fernandez-Vina et al., 1992. Hum Immunol 33:163.
26. Krausa et al., 1995. Tissue Antigens 45:223.
27. Marsh et al., 2000. The HLA Facts Book. Academic Press, San Diego, Calif.
28. Rohrlich et al., 2003. Int Immunol 15:765.
29. Rohrlich et al., 2004, Hum Immunol 65:514.
30. Parker et al., 1994. J Immunol, 152:163.
31. Rammensee et al., 1995. Immunogenetics 41:178.
32. Rammensee et al., 1999. Immunogenetics 50:213.
33. Kuttler et al., 2000. J Mol Biol 298:417.
34. Nussbaum et al., 2001, Immunogenetics 53:87.
35. Sidney et al., 1995. J Immunol 154:247.
36. Theobald et al., 1997. J Exp Med 185:833.
37. Brousset et al., 1998. Mol Pathol 51:170.
38. Burnet, 1971. Transplant Rev 7:3.
39. Hernandez et al., 2004. Eur J Immunol 34:2331.
40. Goulder et al., 1997. J Exp Med 185:1423.
41. Valmori et al., 1999. Int Immunol 11:1971.
42. Valmori et al., 1998. J Immunol 161:6956.
43. Overwijk et al., 1998. J Exp Med 188:277.
44. Slansky et al., 2000. Immunity 13:529.
45. Tangri et al., 2001. J Exp Med 194:833.

We claim:

1. A composition for induction of a cytotoxic T lymphocyte response, comprising: at least one HLA-B7-restricted human telomerase reverse transcriptase (TRT) peptide from nine to twelve amino acid residues in length.

2. The composition of claim 1, wherein said HLA-B7 is selected from the group consisting of HLA-B*0702, HLA-B*3501, HLA-B*3502, HLA-B*3503, HLA-B*5101, HLA-B*5301, HLA-B*5401, HLA-B*0703, HLA-B*0704, HLA-B*0705, HLA-B*1508, HLA-B*5501, HLA-B*5502, HLA-B*5601, HLA-B*5602, HLA-B*6701, HLA-B*7801, and HLA-B*0801.

3. The composition of claim 1, wherein said HLA-B7 is HLA-B*0702.

4. The composition of claim 1, wherein said at least one TRT peptide consists of a sequence selected from the group consisting of SEQ ID NO:3 (p277), SEQ ID NO:4 (p342), SEQ ID NO:6 (p464), SEQ ID NO:8 (p 1107), and SEQ ID NO:9 (p 1123).

5. The composition of claim 1, wherein said at least one TRT peptide consists of a sequence set forth as SEQ ID NO:9 (p1123).

6. The composition of claim 1, further comprising a helper peptide, wherein said TRT peptide is not conjugated to said helper peptide.

7. The composition of claim 1, further comprising an adjuvant.

8. The composition of claim 1, further comprising a physiologically acceptable carrier.

9. The composition of claim 8, wherein said carrier is a mammalian cell.

10. The composition of claim 1, wherein said TRT peptide comprises a modification to enhance binding to HLA-B7.

11. The composition of claim 1, wherein said TRT peptide is a synthetic peptide.

12. A method for inducing or enhancing a CTL response against target cells expressing human TRT and HLA-B7, comprising: harvesting leucocytes expressing HLA-B7; pulsing said leucocytes with the composition of claim 1 comprising an HLA-B7 restricted human TRT peptide; and contacting target cells expressing human TRT and HLA-B7 with said pulsed leucocytes.

13. The method of claim 12, wherein said contacting is accomplished in vitro.

14. The method of claim 12, wherein said contacting is accomplished in vivo.

15. A method for screening HLA class I-restricted human telomerase reverse transcriptase (TRT) peptides, comprising:
a) using an algorithm to identify a human telomerase reverse transcriptase (TRT) peptide sequence in the full length TRT protein sequence that corresponds to a canonical HLA class I motif and comprises at least nine amino acid residues;
b) testing HLA class I binding of said TRT peptide sequence by measuring HLA class I binding or stabilization in comparison to a reference peptide; and
c) assessing immunogenicity of said TRT peptide sequence by measuring induction of TRT peptide-reactive cytotoxic T lymphocytes (CTL) of an HLA class I-positive subject.

16. The method of claim 15, wherein said HLA class I-positive subject was immunized with a human TRT vaccine prior to said assessing of step c).

17. The method of claim 16, wherein said human TRT vaccine comprises human TRT DNA.

18. The method of claim 16, wherein said human TRT vaccine comprises a recombinant microorganism engineered to express human TRT.

19. The method of claim 15, wherein said HLA class I is HLA-B7.

20. The method of claim 15, wherein said HLA class I binding comprises HLA-B*0702 binding, and one or more of HLA-B*3501, HLA-B*3502, HLA-B*3503, HLA-B*5101, HLA-B*5301, HLA-B*5401, HLA-B*0703, HLA-B*0704, HLA-B*0705, HLA-B*1508, HLA-B*5501, HLA-B*5502, HLA-B*5601, HLA-B*5602, HLA-B*6701, HLA-B*7801, and HLA-B*0801 binding.

21. The method of claim 15, wherein said HLA class I is selected from the group consisting of HLA-A3, HLA-A24, HLA-B44, HLA-A1, and HLA-B27.

22. The method of claim 15, wherein said HLA class I-positive subject is a transgenic mouse.

23. A composition for induction of a cytotoxic T lymphocyte response, comprising: at least one HLA class I-restricted human telomerase reverse transcriptase (hTRT) peptide from nine to twelve amino acid residues in length, wherein said hTRT peptide comprises one or more of an HLA-A3-restricted hTRT peptide, an HLA-A24-restricted hTRT peptide, an HLA-B44-restricted hTRT peptide, an HLA-A1-restricted hTRT peptide, and an HLA-B27-restricted hTRT.
